(19) United States
(12) Patent Application Publication
(10) Pub. No.: US 2022/0111110 A1
(43) Pub. Date: Apr. 14, 2022

(54) SANITISER UNIT

(71) Applicant: MECHLINE DEVELOPMENTS LIMITED, Milton Keynes (GB)

(72) Inventor: Peter Galliford, Bedford (GB)

(73) Assignee: MECHLINE DEVELOPMENTS LIMITED, Milton Keynes (GB)

(21) Appl. No.: 17/071,904

(22) Filed: Oct. 15, 2020

Related U.S. Application Data
(63) Continuation-in-part of application No. 29/754,800, filed on Oct. 14, 2020, now Pat. No. D944377.

(51) Int. Cl. A61L 9/20

(52) U.S. Cl. CPC A61L 9/205; A61L 2209/12; A61L 2209/111

(57) ABSTRACT

An air sanitiser unit includes a housing including a base with one or more mounting formations for mounting the base on a substantially vertical wall or support structure and a lid having an air inlet vent and an air outlet vent. The lid is releasably attached to the base to provide an enclosure. At least one ultraviolet lamp is enclosed within the housing to prevent exposure of the ultraviolet lamp from outside of the housing. One or more diffuser elements are enclosed in the housing adjacent to the ultraviolet lamp including one or more photocatalysts. A control device is enclosed in the housing with an alarm device. The control device is electrically connected to the ultraviolet lamp and the alarm device and is configured to control the alarm device according to the status of the ultraviolet lamp.

### CROSS-REFERENCE TO RELATED APPLICATION

This application is a continuation-in-part of U.S. design application Ser. No. 29/754,800 filed Oct. 14, 2020, and the entire disclosure of said design application is hereby expressly incorporated by reference into the present specification.

### FIELD OF INVENTION

The present invention relates to a sanitiser unit for sanitising air and surfaces in the vicinity of the sanitiser unit. The invention also relates to a kit of parts for use with the sanitiser unit.

### BACKGROUND

It is known that air and surfaces can be sanitised by exposure to ultraviolet radiation. In addition, it is known to generate strongly oxidising hydroxyl radicals (OH) by irradiating a photocatalyst material with ultraviolet radiation. These hydroxyl radicals can break down bacteria and pollutants in the air and on surfaces in the vicinity of the sanitiser unit, destroying air-borne pathogens, viruses, bacteria, fungi, mould spores and allergens, and decomposing odours and harmful gases. Exposure to ultraviolet radiation also kills microorganisms (bacteria, viruses and mould) by disrupting their DNA and removing their reproductive capabilities.

Prolonged exposure to ultraviolet radiation can be harmful to human beings. It is important therefore that the ultraviolet radiation is enclosed within a housing so that the radiation is unable to escape from the housing. The housing must however allow air to circulate through the housing, so that pollutants and contaminants in the air can be removed.

The housing should also contain the photocatalyst materials and enable hydroxyl radicals generated by photocatalytic oxidation to flow readily from the housing so that they can continue to destroy contaminants in the air and on surrounding surfaces.

Ultraviolet lamps used in sanitiser units have a limited surface life (typically 8000 hours or 1 year of continuous use). After this time the efficiency of the lamp may have decreased by 20-30%. However, it may not be apparent to a user that the lamp requires replacing. There is a need therefore for a sanitiser unit that indicates to the user when the lamp should be replaced.

The power and operating wavelength of the lamp must also be selected according to the environment in which the sanitiser unit is to be used, and the concentration and type of contaminants it is intended to remove. However, it may not be readily apparent what type of lamp is required when replacing the ultraviolet lamp. There is a need therefore for a sanitiser unit that allows the user to readily identify the correct ultraviolet lamp.

Ultraviolet lamps generally include a quartz glass envelope. If this envelope breaks during use shards of glass may be scattered from the steriliser unit causing a hazard, particularly in food preparation areas. There is a need therefore for a steriliser unit that prevents shards of glass from a broken envelope from escaping.

Steriliser units are generally powered by mains electricity and for safety reasons must be disconnected from the electrical supply when a lamp is replaced. If a user attempts to replace a lamp without disconnecting the electrical supply this may cause a hazard. There is a need therefore for a steriliser unit that ensures the electrical supply is disconnected when a lamp is to be replaced.

Steriliser units may include an electronic control unit, including a memory device that stores information about the unit and its usage. This data may however be lost if the electrical supply is disconnected. Accordingly, a backup battery may be provided to supply electricity to the control unit while the mains electricity supply is disconnected. However, this battery unit may also have a limited service life and will need to be replaced from time to time. Again, it may not be apparent to a user that the battery requires replacing. There is a need therefore for a sanitiser unit that indicates to the user when the battery should be replaced.

### SUMMARY OF THE INVENTION

According to an aspect of the present invention there is provided a sanitiser unit as defined by the appended claims.

According to one embodiment of the present invention there is provided an air sanitiser unit, comprising:
a housing comprising a base with one or more mounting formations for mounting the base on a substantially vertical wall or support structure and a lid having an air inlet vent and an air outlet vent, the lid releasably attached to the base to provide an enclosure;
at least one ultraviolet lamp enclosed within the housing to prevent exposure of the ultraviolet lamp from outside of the housing;
one or more diffuser elements enclosed in the housing adjacent to the ultraviolet lamp comprising one or more photocatalysts;
a control device enclosed in the housing; and
an alarm device;
   wherein the control device is electrically connected to the ultraviolet lamp and the alarm device and is configured to control the alarm device according to the status of the ultraviolet lamp.

Optionally, the ultraviolet lamp has a service life and the control device is configured to activate the alarm device when the service life of the ultraviolet lamp expires. The service life may be a predetermined period of operation, for example 8000 hours, after which the efficiency of the lamp decreases significantly.

Optionally, the control device monitors an operational duration of the ultraviolet lamp and activates the alarm device when the operational duration exceeds the service life of the ultraviolet lamp. For example, the control unit monitors how long the ultraviolet lamp has been operational since it was installed and compares this operational duration with a predetermined value that represents the service life of the ultraviolet lamp. When the operational duration exceeds the predetermined value that represents the service life, the control unit activates the alarm indicating to the user that the lamp should be replaced. The control unit may also be configured to monitor replacement of the ultraviolet lamp and reset the operational duration value when it senses that the ultraviolet lamp has been replaced, or it may be reset by the user using a reset control.

If the sanitiser unit includes a replaceable battery, the control device may be configured to activate the alarm device when the service life of the battery expires, indicating that the battery needs to be replaced.

Optionally, the alarm device comprises a light indicator, for example a flashing light. Optionally, the light indicator comprises a light source and light pipe configured to transmit light from the light source to a hole in the lid.

Optionally, the alarm device comprises an audible sounder, which may be provided as an alternative to the light indicator or in addition to the light indicator.

Optionally, the ultraviolet lamp is replaceable and includes a colour-coded cap proximal to an end of the ultraviolet lamp for indicating at least one characteristic of the ultraviolet lamp and/or the compatibility of the ultraviolet lamp with the housing.

Optionally, the ultraviolet lamp comprises a protective sheath that prevents debris, for example fragments of glass, escaping in the event that the ultraviolet lamp breaks.

Optionally, the protective sheath comprises a material that permits the transmission of ultraviolet light.

Optionally, the air sanitiser unit further comprises an operating indicator configured to indicate that the air sanitiser unit is operating. Optionally, the operating indicator comprises an indicator lamp, for example a green cross, which emits light to indicate that the sanitiser unit is operating.

Optionally, the one or more diffuser elements comprise aluminium diffuser plates carrying the one or more photocatalysts. Optionally, the one or more photocatalysts comprise titanium dioxide (TiO₂).

Optionally, the housing includes shielding elements that prevent exposure of the ultraviolet lamp through the air inlet and outlet vents.

Optionally, the base and the lid include mounting formations that can be engaged for mounting the lid to the base.

Optionally, the air sanitiser unit further comprises a safety switch, wherein the lid is configured to disengage the safety switch when mounted to the base to activate the air steriliser unit, and wherein the lid is configured to engage the safety switch when dismounted from the base to deactivate the air steriliser unit to prevent exposure of the ultraviolet lamp when the lid is removed. Optionally, the safety switch comprises a microswitch.

Optionally, the air sanitiser unit further comprises a power supply, said power supply comprising a connector for connection to a mains electricity supply.

Optionally, the air sanitiser unit further comprises a battery configured to power the control device. The ensures continued operation of the control unit if the mains electricity supply is disconnected. The battery may be rechargeable from the mains electricity supply and/or it may be replaceable.

According to another aspect of the invention there is provided a kit of parts for use with an air sanitiser unit as defined above, the kit of parts comprising at least one replacement ultraviolet lamp, wherein the replacement ultraviolet lamp includes a colour-coded cap proximal to an end of the ultraviolet lamp for indicating at least one characteristic of the ultraviolet lamp and/or the compatibility of the ultraviolet lamp with the housing of the air steriliser.

Optionally, the kit of parts further comprises a replacement battery.

Optionally, the kit of parts further comprises one or more labels for attachment to the housing, the labels denoting ultraviolet lamp size and being colour-coded to the colour-coded cap.

### BRIEF DESCRIPTION OF THE DRAWINGS

An embodiment of the invention will now be described by a way of example with reference to the accompanying drawings, in which:

FIG. 1 is an isometric view of the steriliser unit;

FIG. 2 is a front view of the steriliser unit;

FIG. 3 is a rear view:

FIG. 4 is a left side view:

FIG. 5 is a right side view;

FIG. 6 is a top plan view;

FIG. 7 is a bottom plan view;

FIG. 8 is an exploded isometric view showing the internal components of the steriliser unit;

FIG. 8a is a detail view of a portion of FIG. 8, and

FIG. 9 is a front view of the steriliser unit, showing details that are not apparent in the other drawings.

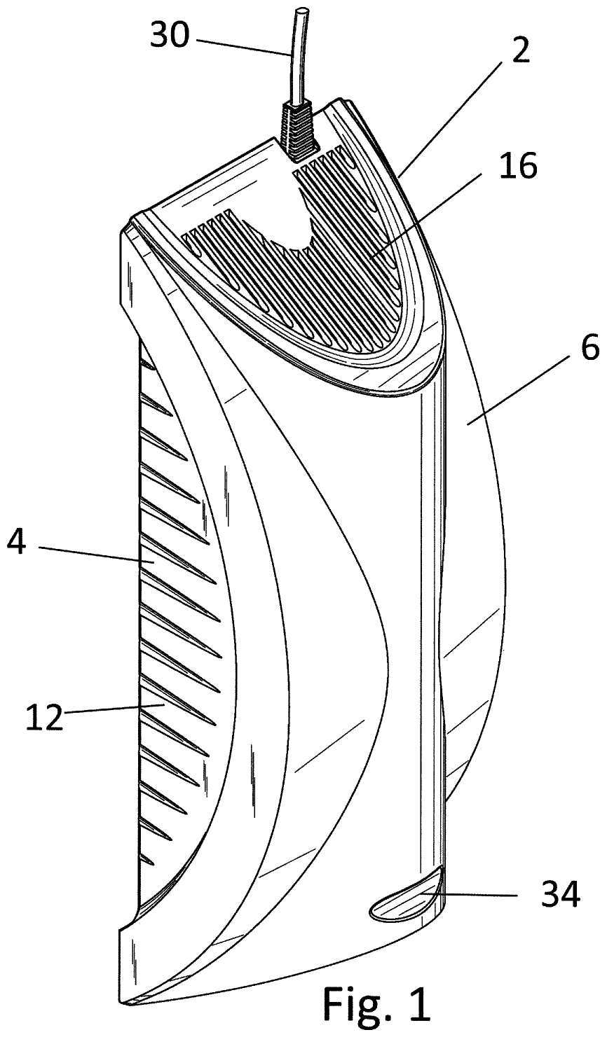

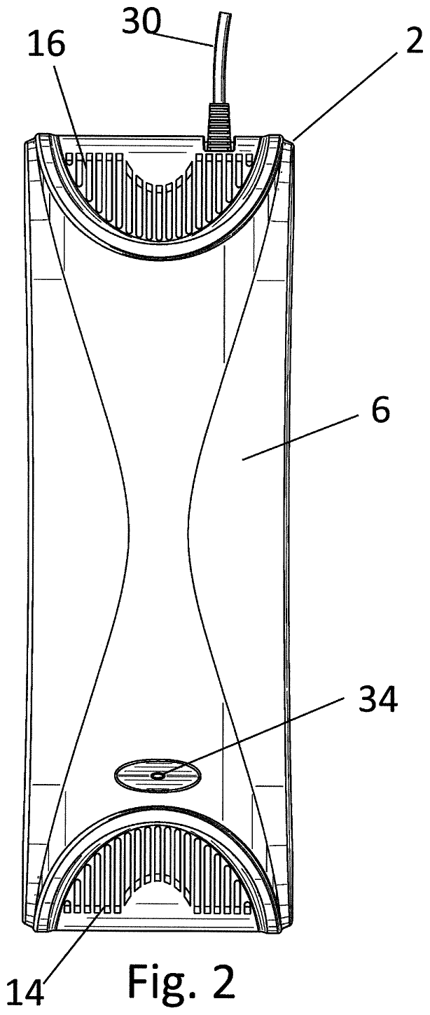

### DETAILED DESCRIPTION OF THE INVENTION

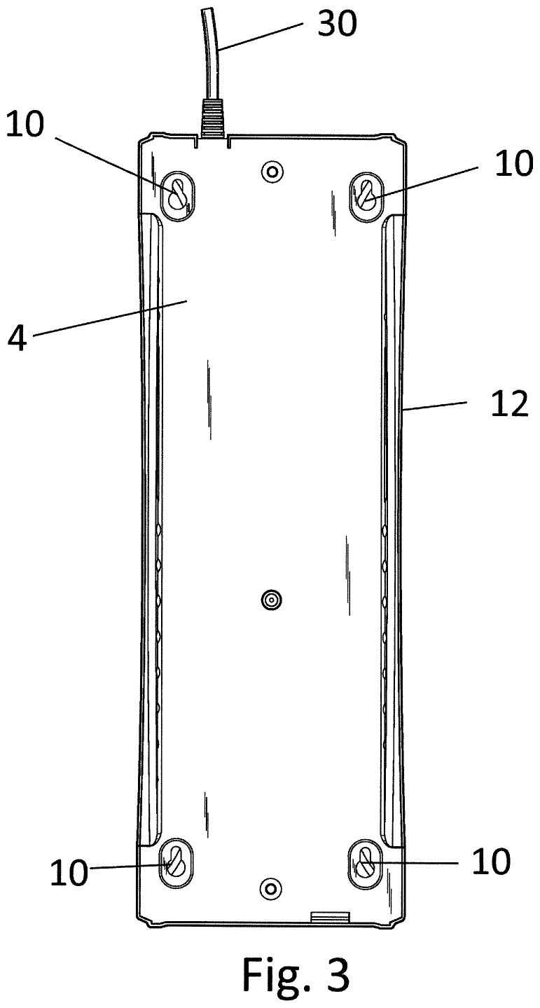

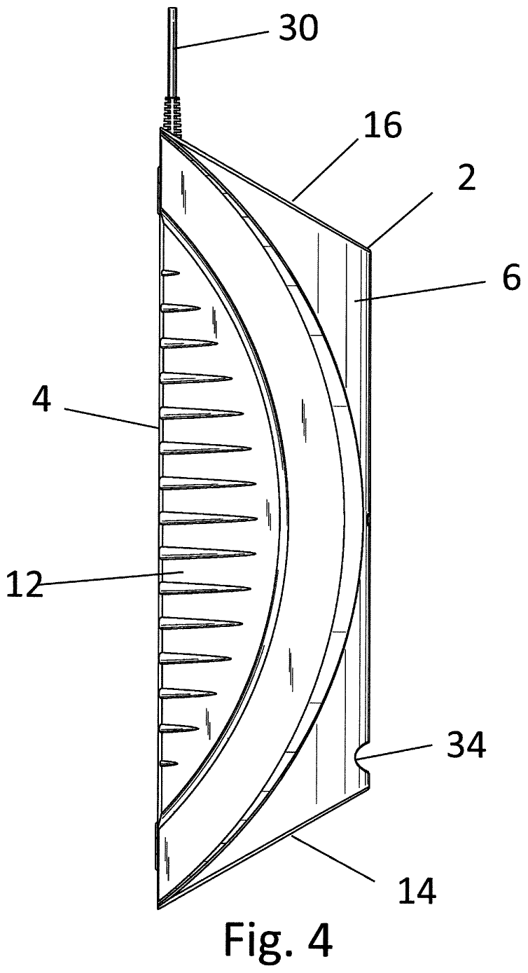

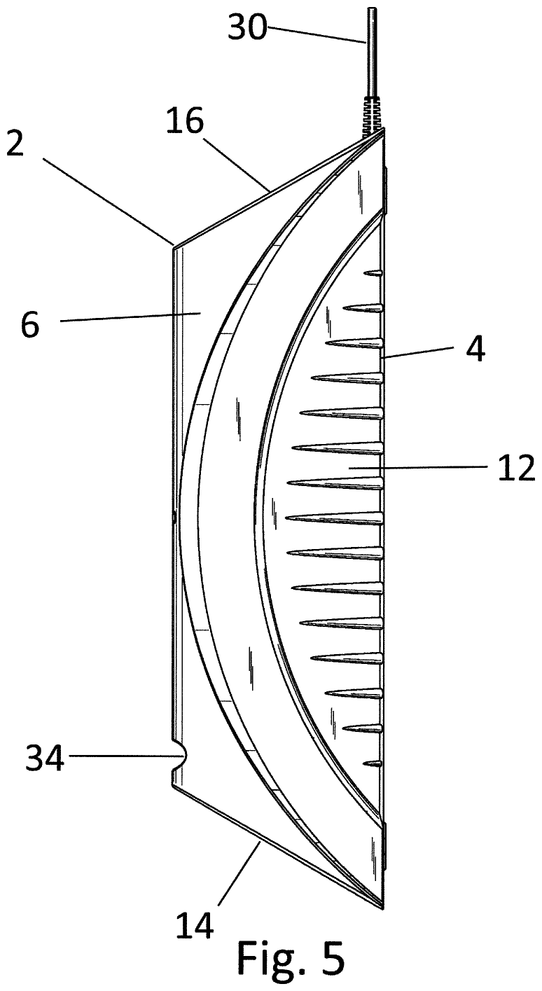

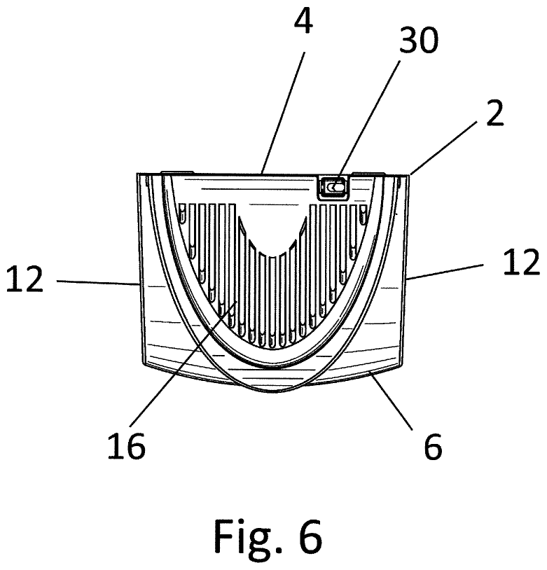

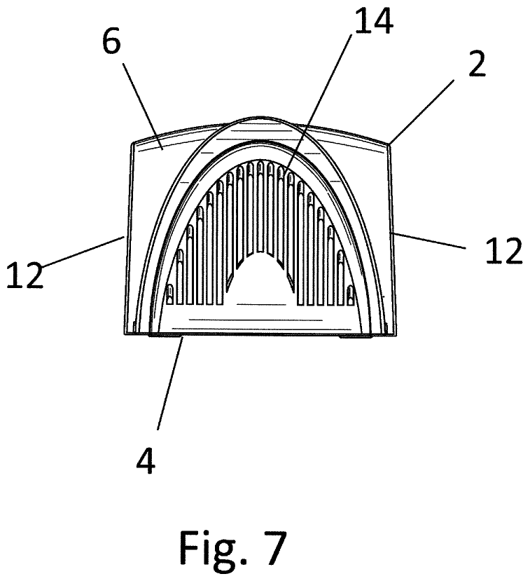

As shown in the accompanying drawings, the steriliser unit includes a housing 2 that comprises a base plate 4 and a lid 6 that may be removably attached to the base plate 4, for example with screws 8. As shown in FIG. 3, the base plate 4 includes four keyhole slots 10 for attaching the steriliser unit to a wall or other surface.

The base plate 4 carries two spaced side walls 12 that extend perpendicular to the base plate. The lid 6 is shaped to engage the base plate 4 at the upper and lower ends thereof, and engages the outer edges of the side plates 12 to form an enclosure. The upper and lower ends of the lid 6 are inclined relative to the plane of the base plate 4 and include openings forming an air inlet vent 14 at the lower end of the housing 2 and an air outlet vent 16 at the upper end of the housing 2, which allow air to flow by convection through the housing 2 driven by the heating effect of the lamp 20.

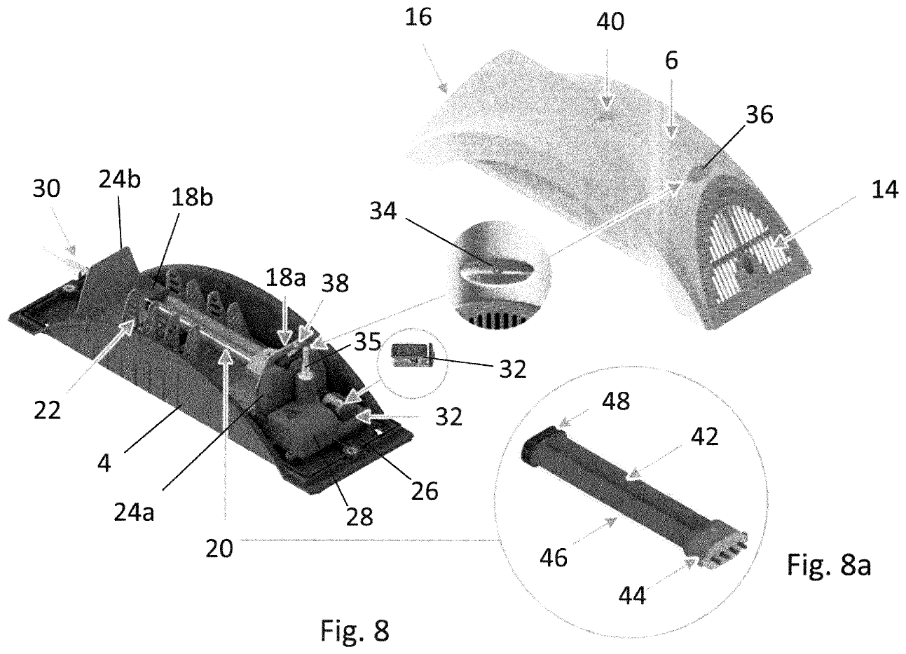

As illustrated in FIG. 8, the base plate 4 includes mounting elements 18a, 18b for an ultraviolet lamp 20. In this embodiment the lower mounting element 18a carries electrical connectors for powering the lamp 20 and the upper mounting element 18b simply supports the lamp 20. However, this arrangement may be reversed.

On either side of the lamp 20 a plurality of air diffuser plates 22 are mounted to the base plate 4 and project outwards therefrom. The air diffuser plates 22 include perforations allowing air to pass through and around the plates as it flows through the housing 2. The air diffuser plates 22 may for example be made of aluminium. The air diffuser plates 22 are coated with a photocatalyst material, for example titanium dioxide TIO₂, which emits hydroxyl radicals (OH) when illuminated with ultraviolet radiation from the lamp 20.

At the ends of the lamp 20, upper and lower shield elements 24a, 24b are provided, which prevent ultraviolet radiation emitted by the lamp 20 from passing through the inlet and outlet air vents 14, 16 at the lower and upper ends of the housing 2. Ultraviolet radiation is thus prevented from escaping the housing 2.

Below the lower shield 24a a compartment 26 is provided, which accommodates an electronic control unit 28. The electronic control unit 28 receives power from a mains electricity input cable 30, which enters the housing 2 at the upper end of the base plate 4. A rechargeable backup battery 32 is charged from the mains electricity supply via the electronic control unit 28 and provides power to the electronic control unit 28 when the mains electricity supply is disconnected. A warning lamp 34, for example an LED lamp is connected to the electronic control unit 28, which is configured to light the lamp 34 after a predetermined number of operating hours corresponding to the service life of the lamp 20 and the batter 32. For example, the electronic control unit 28 may be configured to activate the warning lamp 34 after 8000 hours or 12 months of continuous use. This serves to warn a user that the battery 32 and the ultraviolet lamp 20 should be replaced. The warning lamp 34 is connected to a light pipe 35 that protrudes through a window 36 in the lower part of the lid 6 so that it can be easily seen. Optionally, a sounded device may also be connected to the electronic control unit 28 and configured to provide an audible warning if the ultraviolet lamp 20 and the battery 32 need to be replaced.

A safety switch 38, for example a microswitch, is mounted on the lower shield element 24a and is arranged to be contacted by the lid 6 when it is securely attached to the base plate 4. The safety switch 38 controls operation of the lamp 20 via the electronic control unit 28 and the lid is configured to disengage the safety switch when it is mounted to the base to activate the steriliser unit, and to engage the safety switch when the lid is dismounted from the base to deactivate the steriliser unit, to prevent exposure of the ultraviolet lamp when the lid is removed.

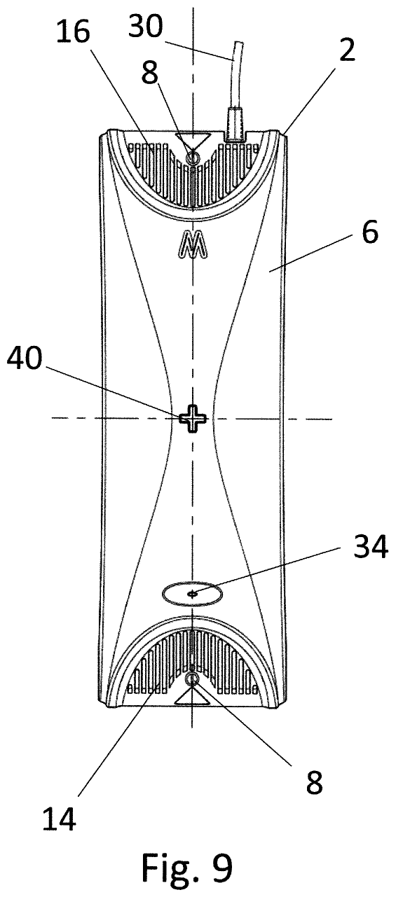

As illustrated in FIGS. 8 and 9, the lid 6 incorporates an indicator lamp 40, which is illuminated when the steriliser unit is operating. The indicator lamp 40 may for example take the form of a green cross.

An ultraviolet lamp 20 for use in the steriliser unit is shown in more detail in FIG. 8a. In this example, the ultraviolet lamp 20 includes two ultraviolet tubes contained within quartz glass envelopes and connected to an electrical end terminal 44. The tubes 44 are contained within a transparent sheath 46, which contains any debris such as glass fragments if either of the tubes breaks.

A colour-coded end cap 48 is provided at the opposite end of the UV lamp 20. The end cap 48 is colour-coded to indicate the power and operating wavelength of the ultraviolet lamp 20. For example, different lamps may be provided according to whether the sanitiser unit is intended for use in occupied areas of different sizes, food preparation areas, refuse areas and so on.

In use, when replacing the ultraviolet lamp, the user can easily identify the correct replacement lamp by referring to the colour coding. The colour coding may also be repeated on the housing, for example on the base plate 4 or the lid 6, for easy reference and to ensure that the correct lamp is used.

1. An air sanitiser unit, comprising:
a housing comprising a base with one or more mounting formations for mounting the base on a substantially vertical wall or support structure and a lid having an air inlet vent and an air outlet vent, the lid releasably attached to the base to provide an enclosure;
at least one ultraviolet lamp enclosed within the housing to prevent exposure of the ultraviolet lamp from outside of the housing;
one or more diffuser elements enclosed in the housing adjacent to the ultraviolet lamp comprising one or more photocatalysts;
a control device enclosed in the housing; and
an alarm device;
wherein the control device is electrically connected to the ultraviolet lamp and the alarm device, and is configured to control the alarm device according to the status of the ultraviolet lamp.

2. The air sanitiser unit according to claim 1, wherein the ultraviolet lamp has a service life and the control device is configured to activate the alarm device when the service life of the ultraviolet lamp expires.

3. The air sanitiser unit according to claim 2, wherein the control device monitors an operational duration of the ultraviolet lamp and activates the alarm device when the operational duration exceeds the service life of the ultraviolet lamp.

4. The air sanitiser unit according to claim 1, wherein the alarm device comprises a light indicator.

5. The air sanitiser unit according to claim 4, wherein the light indicator comprises a light source and light pipe configured to transmit light from the light source to a hole in the lid.

6. The air sanitiser unit according to claim 1, wherein the alarm device comprises an audible sounder.

7. The air sanitiser unit according to claim 1, wherein the ultraviolet lamp is replaceable and includes a colour-coded cap proximal to an end of the ultraviolet lamp for indicating at least one characteristic of the ultraviolet lamp and/or the compatibility of the ultraviolet lamp with the housing.

8. The air sanitiser unit according to claim 1, wherein the ultraviolet lamp comprises a protective sheath that prevents debris escaping in the event that the ultraviolet lamp breaks.

9. The air sanitiser unit according to claim 8, wherein the protective sheath comprises a material that permits the transmission of ultraviolet light.

10. The air sanitiser unit according to claim 1, further comprising an operating indicator configured to indicate that the air sanitiser unit is operating.

11. The air sanitiser unit according to claim 10, wherein the operating indicator comprises an indicator lamp, which emits light to indicate that the sanitiser unit is operating.

12. The air sanitiser unit according to claim 1, wherein the one or more diffuser elements comprise aluminium diffuser plates carrying the one or more photocatalysts.

13. The air sanitiser unit according to claim 1, wherein the one or more photocatalysts comprise titanium dioxide.

14. The air sanitiser unit according to claim 1, wherein the housing includes shielding elements that prevent exposure of the ultraviolet lamp through the air inlet and outlet vents.

15. The air sanitiser unit according to claim 1, wherein the base and the lid include mounting formations that can be engaged for mounting the lid to the base.

16. The air sanitiser unit according to claim 1, further comprising a safety switch, wherein the lid is configured to disengage the safety switch when mounted to the base to activate the air steriliser unit, and wherein the lid is configured to engage the safety switch when dismounted from the base to deactivate the air steriliser unit to prevent exposure of the ultraviolet lamp when the lid is removed.

17. The air sanitiser unit according to claim 16, wherein the safety switch comprises a microswitch.

18. The air sanitiser unit according to claim 1, further comprising a power supply, said power supply comprising a connector for connection to a mains electricity supply.

19. The air sanitiser unit according to claim 1, further comprising a battery configured to power the control device.

20. A kit of parts for use with an air sanitiser unit according to claim 1, the kit of parts comprising a replacement ultraviolet lamp, wherein the ultraviolet lamp includes a colour-coded cap proximal to an end of the ultraviolet lamp for indicating at least one characteristic of the ultraviolet lamp and/or the compatibility of the ultraviolet lamp with the housing of the air steriliser.

21. The kit of parts according to claim 20, further comprising a replacement battery.

22. The kit of parts according to claim 20, further comprising one or more labels denoting ultraviolet lamp size and colour-coded to the colour-coded cap.
